(19) United States
(12) Patent Application Publication
(10) Pub. No.: US 2023/0326593 A1
(43) Pub. Date: Oct. 12, 2023

(54) Remote Medical Consultation System and Method Adapted for People with Reduced Mobility or Suffering from Visual or Verbal Disabilities

(71) Applicant: DR H. NICOLAS INC., Laval (CA)

(72) Inventor: Hanna Nicolas, Laval, QC (CA)

(21) Appl. No.: 18/296,835

(22) Filed: Apr. 6, 2023

Related U.S. Application Data
(60) Provisional application No. 63/362,739, filed on Apr. 8, 2022.

(51) Int. Cl. G16H 40/67; G16H 80/00; A61L 2/10; A61L 2/20; A61L 2/24

(52) U.S. Cl. CPC G16H 40/67; G16H 80/00; A61L 2/10; A61L 2/20; A61L 2/24

(57) ABSTRACT

A remote medical consultation system and method comprising a telecommunication platform adapted for people with physical, motor, visual and/or verbal disabilities. The remote medical consultation system provides an environment adapted for patients with reduced mobility and further comprises medical diagnostic equipment.

### CROSS-REFERENCE TO RELATED APPLICATIONS

The present patent application claims the benefits of priority of U.S. Patent Application No. 63/362,739, entitled “Remote Medical Consultation System and Method Adapted for People with Reduced Mobility or Suffering from Visual or Verbal Disabilities”, and filed at the USPTO on Apr. 8, 2022, the content of which is incorporated herein by reference.

### FIELD OF THE INVENTION

The present invention generally relates to the field of medical consultation systems and method. More specifically, the present invention relates to vertical medical consultation systems and methods for patients with disabilities.

### BACKGROUND OF THE INVENTION

The COVID-19 pandemic has had a severe impact on healthcare systems and has stretched several services passed their breaking point. These problems have led to a broad adoption of virtual medical consultations.

However, current systems and methods of providing virtual medical consultations can be frustrating to use for providers and patients alike. Many users struggle with complex devices and functions which create barriers to their acceptance and adoption. In particular, elderly patients and patients with reduced mobility and disabilities are disproportionately affected.

There is therefore a need for a system and method of providing virtual medical consultations offering a safe, secure, and user-friendly way to connect patients with reduced mobility and/or verbal or visual disability in urban, rural and remote communities while offering high-quality primary care.

### SUMMARY OF THE INVENTION

The shortcomings of the prior art are generally mitigated by a remote medical consultation system comprising a telecommunication platform adapted for people with physical, motor, visual and/or verbal disabilities. The remote medical consultation system provides an environment adapted for patients with reduced mobility and further comprises medical diagnostic equipment.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the accompanying drawings in which:

FIG. 1 is a front view of the remote medical consultation system in accordance with the principles of the present invention.

FIG. 2 is a side view of the remote medical consultation system of FIG. 1.

FIG. 3 is a rear view of the remote medical consultation system of FIG. 1.

FIG. 4 is a top plan view of the interior of the remote medical consultation system of FIG. 1.

FIG. 5 is a front view of the of the interior of the remote medical consultation system of FIG. 1.

FIG. 6 is a side view of the of the interior of the remote medical consultation system of FIG. 1.

FIG. 7 is another top plan view of an interior of the remote medical consultation system of FIG. 1.

FIG. 8 is a top plan view of the ceiling of the remote medical consultation system of FIG. 1.

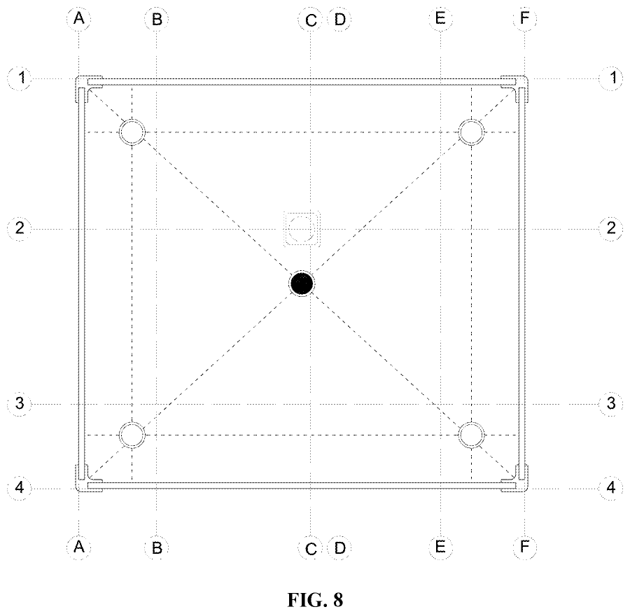

FIG. 9 is a top plan view of the interior of the remote medical consultation system of FIG. 1 illustrating various positions of a patient during the arrival and entry into the remote medical consultation system.

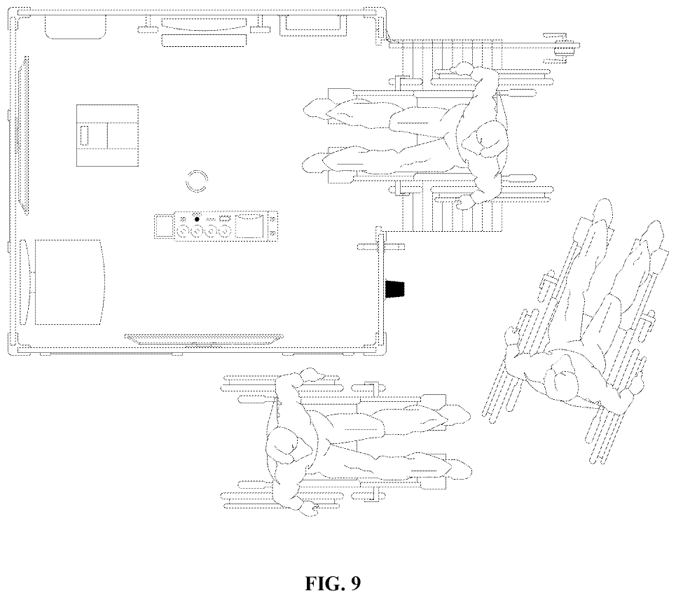

FIG. 10 is a top plan view of the interior of the remote medical consultation system of FIG. 1 illustrating a patient positioned therein.

FIG. 11 is a top plan view of the interior of the remote medical consultation system of FIG. 1 illustrating various positions of a patient during their positioning onto a chair.

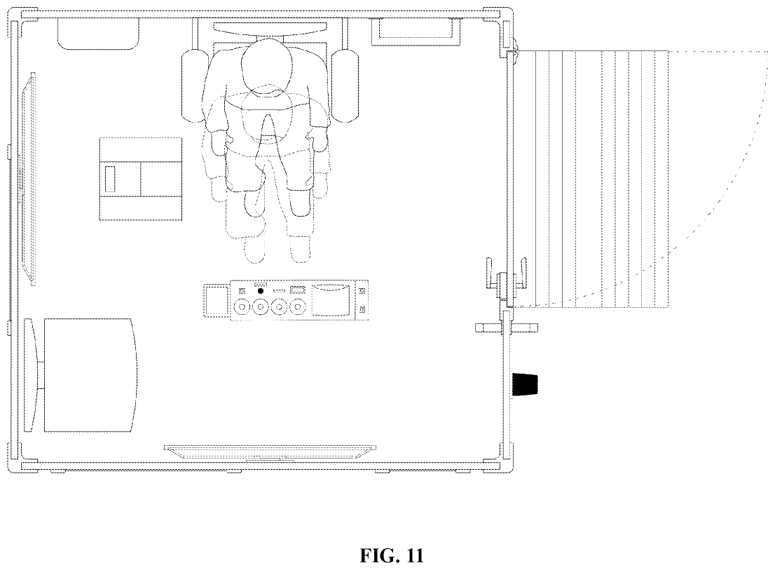

### DETAILED DESCRIPTION OF THE INVENTION

A novel remote medical consultation system and method will be described hereinafter. Although the invention is described in terms of specific illustrative embodiments, it is to be understood that the embodiments described herein are by way of example only and that the scope of the invention is not intended to be limited thereby.

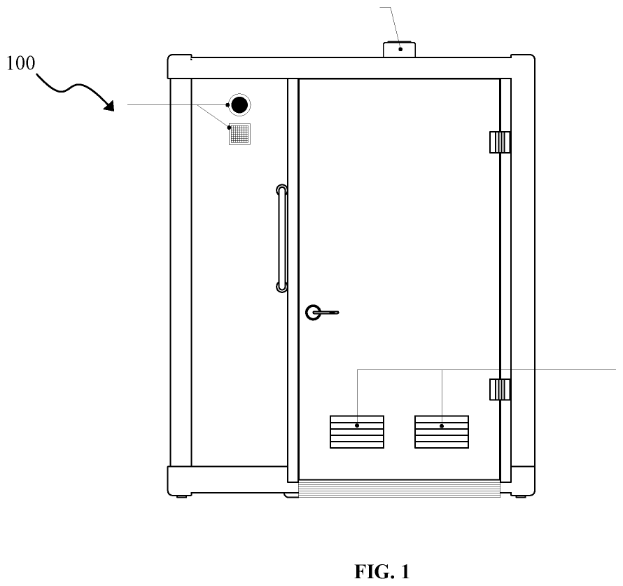

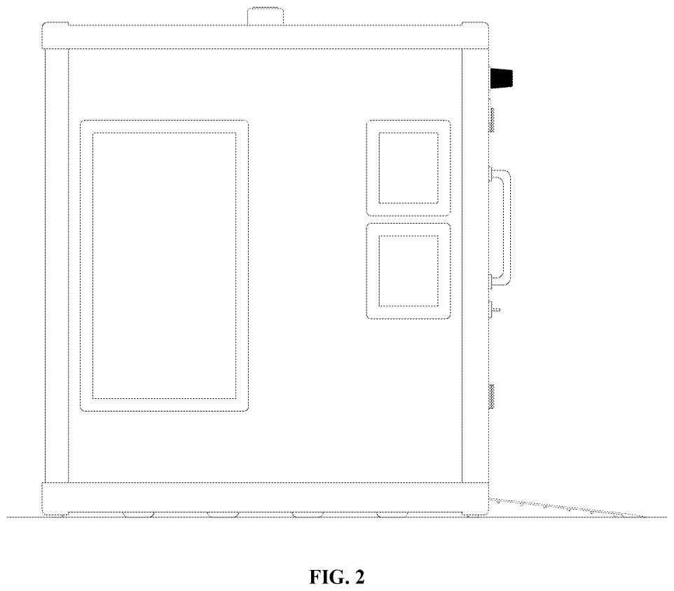

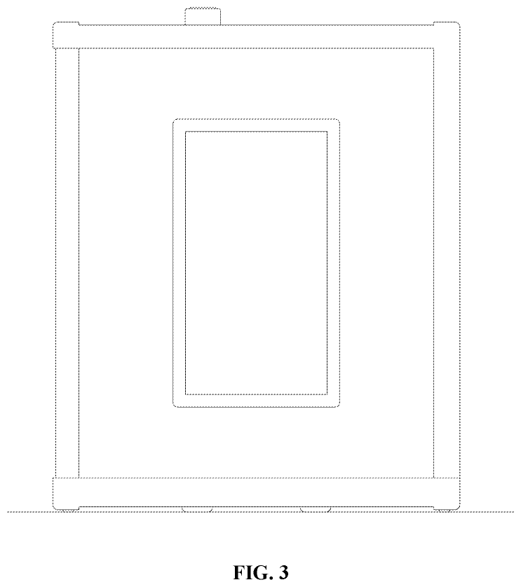

Referring to FIGS. 1 to 3, a remote medical consultation system 100 is illustrated. Broadly, the remote medical consultation system 100 is equipped with a video terminal, wireless communications capabilities and may be maneuvered to remote areas providing medical care in geographic regions not capable of supporting independent medical personnel.

The remote medical consultation system 100 comprises an enclosure 26 and a door 15 with a latch for allowing entry or egress of a patient therefrom. The remote medical consultation system 100 may further comprise a ramp 14 to allow access to patients with reduced mobility. The door 15 may be adapted for people with reduced mobility. In particular, the door 15 may open with an external wall button and may be closed automatically or using the latch. To that end, the remote medical consultation system 100 may comprise a button for activating an opening of the door 15. In certain embodiments, the door 15 may be actuated remotely by the medical practitioner.

The remote medical consultation system 100 may enable patients to remotely consult with practitioners for a variety of common outpatient symptoms. As described in greater detail below, the remote medical consultation system 100 generally comprises a plurality of medical devices to be used by the patient as instructed by the medical practitioner. Understandably, remote medical consultation system 100 may allow early detection and treatment of illness as well as reduced associated outpatient care costs.

The remote medical consultation system 100 comprises bi-directional communication platform configured to carry data for a digital video conference between a patient and a health care provider. Furthermore, all data and information collected or retrieved by the medical devices may be automatically sent through the communication platform. The communication platform may further comprise a WiFi and/or Bluetooth system adapted to provide a WiFi connection to the medical devices and/or a phone or other similar device operated by the patient.

In certain embodiments, the virtual medical office may be small enough that it could be permanently or temporarily installed in a desired location such as, but not limited to, a supermarket, a grocery store, a pharmacy or elderly home. It may also be mobile.

In certain embodiments, the remote medical consultation system 100 may provide an acoustically insulated sound reducing enclosure thereby providing a desirable level of privacy during a video consultation.

Moreover, the remote medical consultation system 100 may be pre-sanitized to provide a sanitary environment for patients. For example, the remote medical consultation system 100 may comprise a ventilation system for circulating the air therein. The remote medical consultation system 100 may further comprise a sanitary spray dispersal system configured to discharge a sanitary spray within the remote medical consultation system 100 prior to arrival of a patient or subsequent to their departure. The remote medical consultation system 100 may further comprise a UVC light to further promote sanitation after each patient. The UVC light may be similarly activated subsequent to the departure of a patient.

In certain embodiments, the communication platform of the remote medical consultation system 100 may be linked to local medical emergency departments. Accordingly, a patient or a medical practitioner may alert said medical emergency departments if the patient is in need of immediate medical assistance. To that end, the remote medical consultation system 100 may comprise an emergency or panic button to alert said emergency departments.

The remote medical consultation system 100 may comprise an input platform allowing a patient to identify themselves and to provide any necessary additional information. The input platform may comprise a touch screen, a keyboard, a visual screening interface, a code scanner, a card scanner or any other suitable platform. The additional information may comprise their contact information, emergency contact information, personal details, insurance information, corporate account ID, individual account ID, or any other relevant information requested by the remote medical consultation system 100 or a medical practitioner. In certain embodiments, the patient may be asked to provide information in response to a paper or electronic questionnaire.

As specified above, the remote medical consultation system 100 may comprise medical devices and/or diagnostic devices such as, but not limited to, devices configured to measure blood pressure, cardiac frequency, blood oxygen level, height, weight, BMI, temperature, blood sugar, devices configured to perform a visual acuity test, dermoscopy, hearing test, EKG, a stethoscope, otoscope, PTZ camera, an oximeter, a dermoscope, an ophthalmoscope, an ECG, a tongue depressor device system, a glucometer, a digital scale and/or a digital meter.

Prior to entering the remote medical consultation system 100, a patient may request or initiate a consultation via a terminal located outside the remote medical consultation system 100.

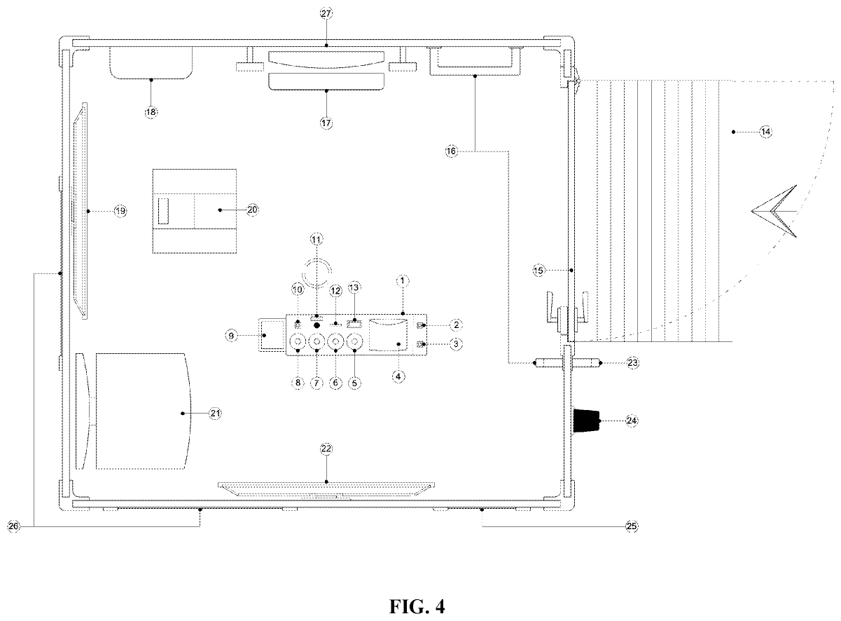

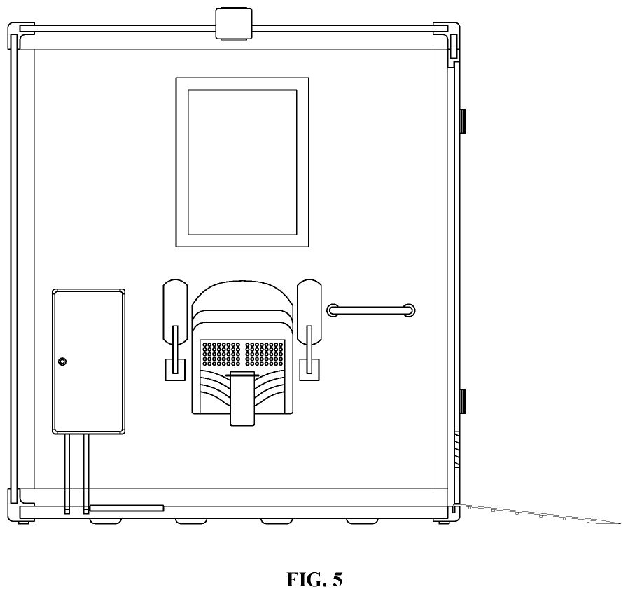

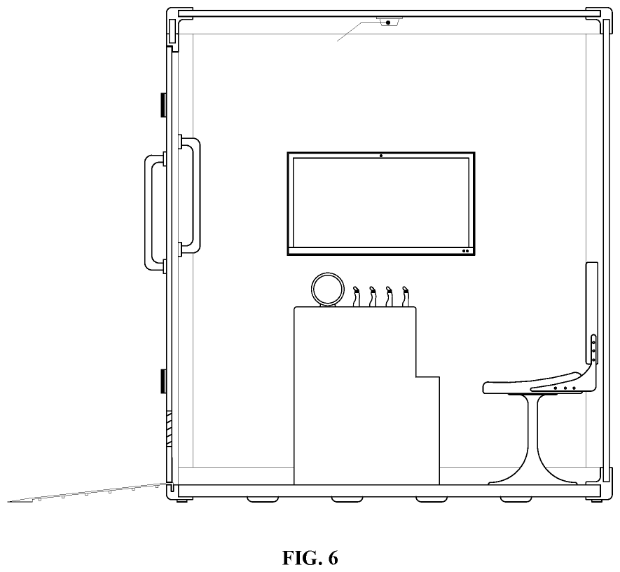

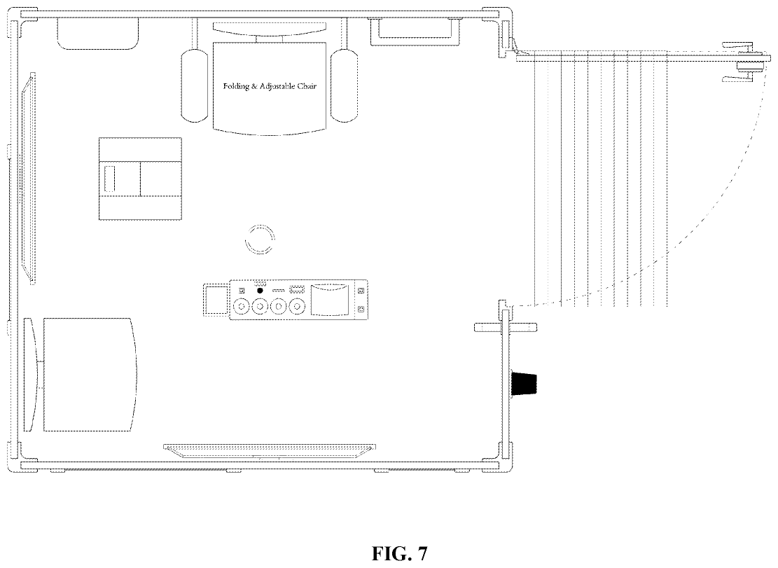

Referring to FIGS. 4 to 7, the remote medical consultation system 100 may comprise a foldable chair 27 configured to receive an ambulant patient. In certain embodiments, the aforementioned medical devices and input platform may be disposed on a panel 1 disposed in front of the foldable chair 27. The remote medical consultation system 100 may further comprise a display 22 where the patient may view and interact with the medical practitioner. In certain embodiments, the remote medical consultation system 100 may further comprise a second display 19 located on a wall adjacent to the display 22 allowing another diagnostic approach.

The remote medical consultation system 100 may further comprise one or more camera modules adapted to provide the medical practitioner a view of the patient within or outside of the remote medical consultation system 100. The camera modules may be located near the displays 19, 22, within the panel 1 or in any other suitable location.

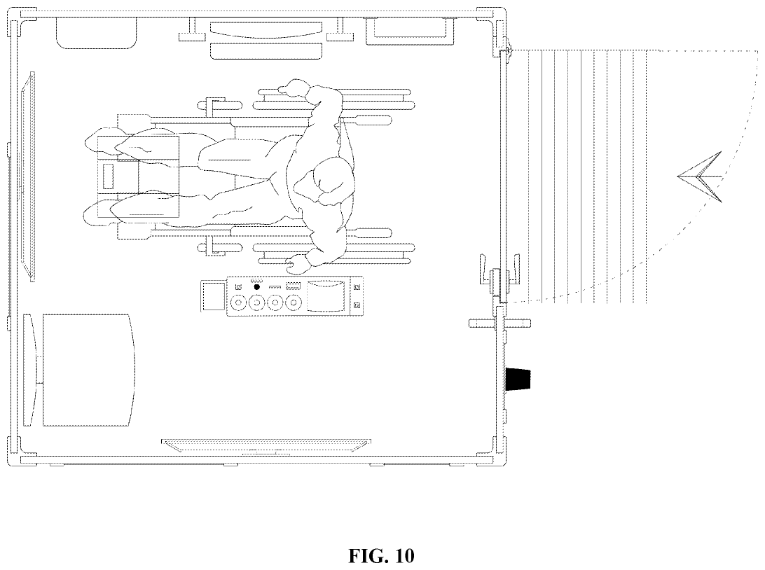

Referring now to FIG. 10, when receiving a patient suffering from a visual, verbal or motor handicap, it may be preferable for the patient to be positioned within the remote medical consultation system 100 while facing the second display 19 which will allow them to easily interact with the medical practitioner. In this embodiment, the panel 1 would be located on their left side. On this same side, the display 22 may allow additional diagnostic assistance.

In certain embodiments, the remote medical consultation system 100 may comprise a companion chair 21 to provide a seating location for an individual accompanying the patient.

At any time during or after the consultation, the medical practitioner may contact local emergency system if necessary, prescribe diagnostic tests, prescribe medication and/or direct the patient to the closest medical center.

In certain embodiments, the remote medical consultation system 100 may be connected to a server configured to store all the information related to the consultation. This data may be viewed by the patient on an online secure platform.

The remote medical consultation system 100 may be connected to the power grid during the entirety of its operation. In certain embodiments, the remote medical consultation system 100 may further comprise an energy storage module configured to power the remote medical consultation system 100 when disconnected from the power grid.

The remote medical consultation system 100 may further comprise an alarm system with siren 24 configured to be activated in the event of an unauthorized entry within the remote medical consultation system 100. Alternatively, the alarm system with siren 24 or other notification device may be activated to alert individuals outside the remote medical consultation system 100 if the patient is in need of immediate assistance. The remote medical consultation system 100 may further comprise an occupancy system configured to visually indicate whether the remote medical consultation system 100 is presently occupied by a patient, unavailable due to maintenance or vacant and ready for use.

A method of providing a remote medical consultation is provided which comprises the following steps:
installing a remote medical consultation system as claimed in claim 1 at a predetermined remote location away from the location of the health care provider;
identifying the patient;
allowing the patient to enter the enclosure and to sit in the chair;
initiating a two-way communication between the health care provider and the patient using the bi-directional communication platform;
selecting a first medical device from said plurality of medical devices;
instructing the patient on the appropriate operation of the first medical device;
transferring the data generated during the operation of the first medical device to the health care provider via the bi-directional communication platform;
selecting an other medical device from said plurality of medical devices;
instructing the patient on the appropriate operation of the other medical device;
transferring the data generated during the operation of the other medical device to the health care provider via the bi-directional communication platform;
repeating the 3 preceding steps until all of the medical devices selected by the health care provider have been operated;
terminating the medical consultation;
allowing the patient to exit the enclosure.

The method may also include one or more of the following steps:
remotely operating the door,
sanitizing the enclosure prior to the arrival of the patient using the sanitary spray dispersal system configured to discharge a sanitary spray within the enclosure,
sanitizing the enclosure prior to the arrival of the patient using a UVC light,
in the event of an emergency, using the communication platform to alert a pre-determined a local medical emergency department of the emergency.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

1. A remote medical consultation system comprising:
an enclosure comprising:
a door adapted to allow the entry into and egress from the enclosure by a patient with reduced mobility,
an area for receiving a chair,
a plurality of medical devices to be used by the patient as instructed by a health care provider located remotely from the enclosure,
a bi-directional communication platform configured to carry data for a digital video and/or voice conference between the patient and the health care provider and data from said medical devices.

2. A remote medical consultation system as claimed in claim 1, wherein the communication platform is linked to a local medical emergency department and comprises an emergency sub-system allowing the patient or the health care provider to alert said medical emergency department if the patient is in need of immediate assistance.

3. A remote medical consultation system as claimed in claim 2, wherein the emergency sub-system comprises a panic button.

4. A remote medical consultation system as claimed in claim 1, wherein the medical consultation system comprises an input platform allowing the patient to identify him/her self and to provide any additional information requested by the health care provider.

5. A remote medical consultation system as claimed in claim 4, wherein the input platform comprises a touch screen, a keyboard, a visual screening interface, a code scanner, a card scanner and/or any other suitable device allowing the patient to interface with the health care provider.

6. A remote medical consultation system as claimed in claim 1, wherein the enclosure is mobile.

7. A remote medical consultation system as claimed in claim 1, wherein the chair is a wheel chair.

8. A remote medical consultation system as claimed in claim 1, wherein the medical devices include two or more devices selected from the following group: device to measure blood pressure, device to measure cardiac frequency, device to measure blood oxygen level, device to measure height, device to measure weight, device to measure BMI, device to measure temperature, device to measure blood sugar, visual acuity testing device, dermoscopy device, hearing testing device, EKG measuring device, a stethoscope, an otoscope, a PTZ camera, an oximeter, a dermoscope, an ophthalmoscope, an ECG measuring device, a tongue depressor device system, a glucometer, a digital scale and a digital meter.

9. A remote medical consultation system as claimed in claim 1, wherein the enclosure is acoustically insulated.

10. A remote medical consultation system as claimed in claim 1, further comprising a sanitary spray dispersal system configured to discharge a sanitary spray within the enclosure prior to arrival of the patient or subsequent to his/her departure.

11. A remote medical consultation system as claimed in claim 10, further comprising a UVC light to further promote sanitation.

12. A remote medical consultation system as claimed in claim 1, further comprising an alarm system with a siren configured to be activated in the event of an unauthorized entry within the remote medical consultation system.

13. A method of providing a virtual medical consultation comprising the following steps:
a. installing a remote medical consultation system as claimed in claim 1 at a predetermined remote location away from the location of the health care provider;
b. identifying the patient;
c. allowing the patient to enter the enclosure and to sit in the chair;
d. initiating a two-way communication between the health care provider and the patient using the bi-directional communication platform;
e. selecting a first medical device from said plurality of medical devices;
f. instructing the patient on the appropriate operation of the first medical device;
g. transferring the data generated during the operation of the first medical device to the health care provider via the bi-directional communication platform;
h. selecting an other medical device from said plurality of medical devices;
i. instructing the patient on the appropriate operation of the other medical device;
j. transferring the data generated during the operation of the other medical device to the health care provider via the bi-directional communication platform;
k. repeating steps g. to i. until all of the medical devices selected by the health care provider have been operated;
l. terminating the medical consultation;
m. allowing the patient to exit the enclosure.

14. A method of providing a virtual medical consultation as claimed in claim 13, further comprising the following step: remotely operating the door.

15. A method of providing a virtual medical consultation as claimed in claim 13, further comprising the following step: sanitizing the enclosure prior to the arrival of the patient using the sanitary spray dispersal system configured to discharge a sanitary spray within the enclosure.

16. A method of providing a virtual medical consultation as claimed in claim 13, further comprising the following step: sanitizing the enclosure prior to the arrival of the patient using a UVC light.

17. A method of providing a virtual medical consultation as claimed in claim 13, further comprising the following step: in the event of an emergency, using the communication platform to alert a pre-determined a local medical emergency department of the emergency.
